(19) United States
(12) Patent Application Publication
(10) Pub. No.: US 2026/0109682 A1
(43) Pub. Date: Apr. 23, 2026

(54) NOVEL COMPOUNDS AND THE USE FOR PROMOTING HAIR GROWTH OR INHIBITING INFLAMMATORY DISORDERS THEREOF

(71) Applicants: Chang Gung University of Science and Technology, Taoyuan City (TW); Kaohsiung Medical University, Kaohsiung City (TW)

(72) Inventors: Tsong-Long Hwang, Taoyuan City (TW); Hsun-Shuo Chang, Kaohsiung City (TW); Chia-Hung Yen, Kaohsiung City (TW); Yu-Chun Lin, Taoyuan City (TW); Tzu-Peng Cheng, Taoyuan City (TW); Ya-Lun Chen, Miaoli County (TW); Ching-Ju Yang, Kaohsiung City (TW)

(73) Assignees: Chang Gung University of Science and Technology, Taoyuan City, TW (TW); Kaohsiung Medical University, Kaohsiung City, TW (TW)

(21) Appl. No.: 18/920,975

(22) Filed: Oct. 20, 2024

(51) Int. Cl. C07D 311/96; A61K 31/352; A61K 36/61; A61P 11/00; A61P 17/14; C07D 311/74; C07D 401/00

(52) U.S. Cl. CPC C07D 311/96; A61K 31/352; A61K 36/61; A61P 11/00; A61P 17/14; C07D 311/74; C07D 401/00

(57) ABSTRACT

Novel compounds and their use for promoting hair growth or inhibiting inflammatory disorders thereof are provided, the compounds have a structure of a formula (I) or a formula (II) or a formula (III), and the compounds or their pharmaceutically acceptable salts can be used for the manufacture of pharmaceutical composition for promoting hair growth or inhibiting inflammatory disorders, the inflammatory disorders including acute respiratory distress syndrome or acute liver injury.

### FIELD OF THE INVENTION

The present invention is related to novel compounds and the use for promoting hair growth or inhibiting inflammatory disorders thereof; In particular, to compounds comprising formula (I), formula (II), or formula (III) and the use for promoting hair growth or inhibiting inflammatory disorders thereof.

### BACKGROUND OF THE INVENTION

Alopecia can be categorized into different patterns of hair loss, including diffuse, specific pattern, and localized. Additionally, it can be further classified based on damage to hair follicle cells into two types: scarring and non-scarring.

Scarring alopecia typically refers to pathological changes or necrosis of scalp hair follicles due to injury or disease, resulting in permanent scars. Common causes include infection, trauma, or illness. Non-scarring alopecia, on the other hand, refers to hair follicles whose regenerative centers remain intact, retaining the ability to produce new hair. Common types of non-scarring alopecia include male pattern baldness, alopecia areata, telogen effluvium, anagen effluvium, secondary syphilis, trichotillomania, and more.

The liver is one of the most important organs in the human body; it can be damaged by drugs, alcohol, diet, and poor work and rest. Liver injury can result in degeneration, necrosis, fibrous tissue hyperplasia, and other lesions of hepatocytes, leading to a series of liver pathological changes and secondary diseases, such as hepatitis, liver cirrhosis, and liver cancer.

Glutamic oxaloacetic transaminase (GOT) and glutamic pyruvic transaminase (GPT) are primarily responsible for metabolizing amino acids and proteins in the human body. Due to their high concentration in liver cells, if the liver is damaged, GOT and GPT can enter the bloodstream. As a result, they are used as biological markers to detect liver injury in blood tests.

Acute Respiratory Distress Syndrome (ARDS) is a severe inflammatory lung condition that impairs the normal function of the alveoli, leading to inadequate oxygen levels in the blood. This can cause damage to other organs and is potentially fatal. Treatment for ARDS primarily involves supportive care, including the use of mechanical ventilators.

These diseases continue to affect many people today, and research into alternative or more effective drugs is ongoing.

Natural compounds are valuable due to their plentiful supply and diverse structures, and they serve as important foundations for drug development. Between 1981 and 2019, nearly half of the new FDA-approved drugs were either derived from natural products or their derivatives. Examples include vincristine, doxorubicin, and paclitaxel for cancer treatment, as well as penicillin derived from fungi for use as antibiotics. The current research is actively focused on identifying which natural compounds have the potential to be developed into new drugs for promoting hair growth, protecting the liver, and preventing ARDS.

### BRIEF SUMMARY OF THE INVENTION

The present invention is a compound having a skeleton comprising a formula (I):

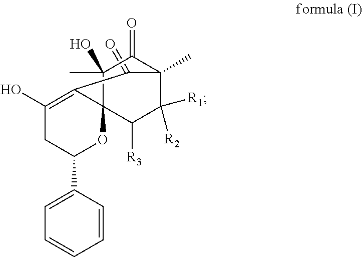

wherein, while R₃ is hydrogen (H), R₁ and R₂ together form a sesquiterpenoid; while R₂ is C1-C6 alkyl group, R₁ and R₃ together form a sesquiterpenoid.

Preferably, the compound of the present invention with a formula (I) includes

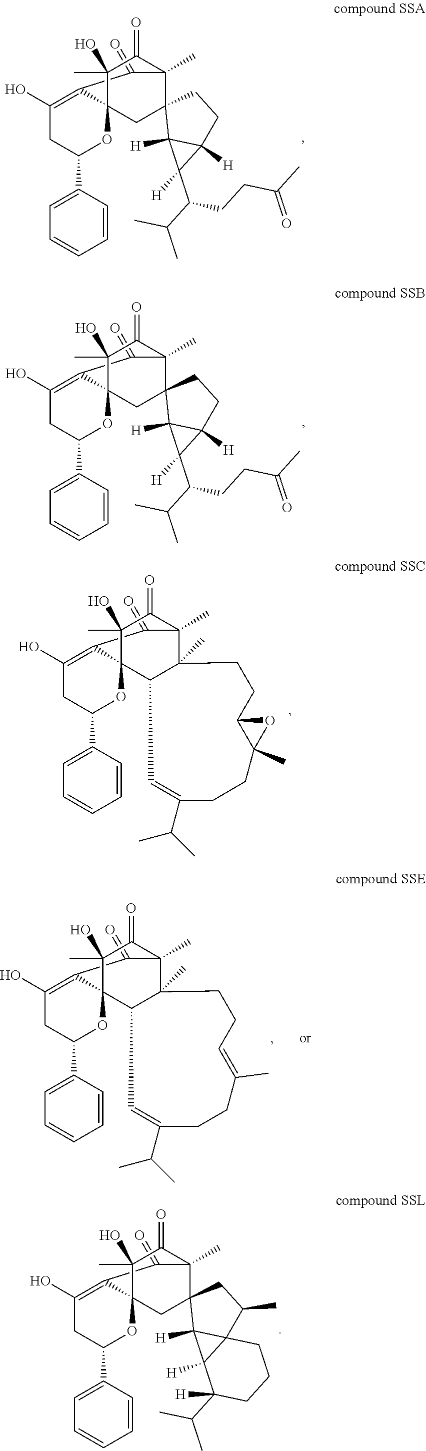

The present invention is related to a compound having a skeleton comprising a formula (II):

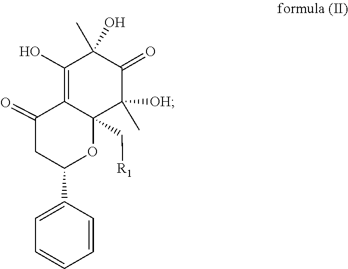

wherein, R₁ is a sesquiterpenoid.

Preferably, the compound of the present invention with a formula (II) is

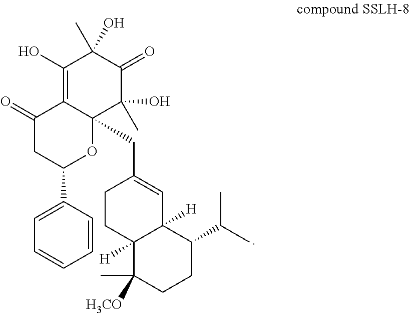 [SMILES: CO[C@]1(C)CC[C@@H](C(C)C)[C@@H]2C=C(C[C@]34O[C@H](c5ccccc5)CC(=O)C3=C(O)[C@](C)(O)C(=O)[C@]4(C)O)CC[C@@H]21]

The present invention is related to a compound having a skeleton comprising a formula (III):

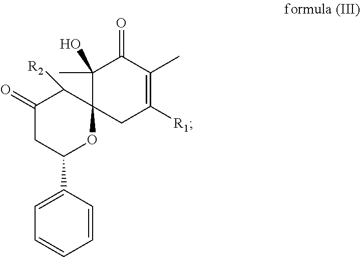

wherein, R₁ is a sesquiterpenoid, R₂ is hydrogen (H), acetoxy group, acetyl group, hydroxy group, methyl group, formyl group, formic acid group, methyl ester group, or halogen.

Preferably, the compound of the present invention with a formula (III) is

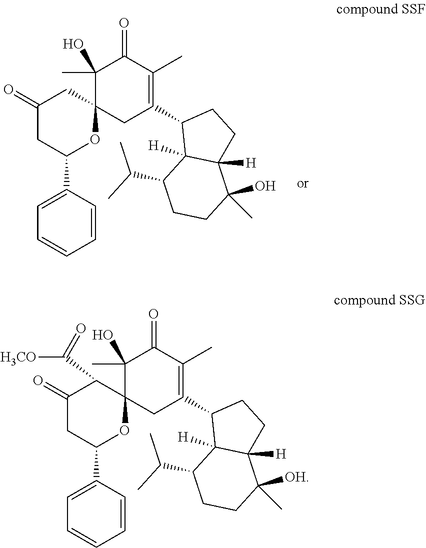 [SMILES: CC1=C([C@@H]2CC[C@H]3[C@H]2[C@@H](C(C)C)CC[C@]3(C)O)C[C@]2(CC(=O)C[C@@H](c3ccccc3)O2)[C@@](C)(O)C1=O ; COC(=O)[C@H]1C(=O)C[C@@H](c2ccccc2)O[C@@]12CC([C@@H]1CC[C@H]3[C@H]1[C@@H](C(C)C)CC[C@]3(C)O)=C(C)C(=O)[C@]2(C)O]

### DETAIL DESCRIPTION OF DRAWINGS

The patent or application file contains at least one drawing executed in color. Copies of this patent or patent application publication with color drawing(s) will be provided by the Office upon request and payment of the necessary fee.

FIG. 1 shows the chemical structure of the new compounds in the present invention.

FIG. 2 shows the results of the novel compound of the present invention at different concentrations inhibiting superoxide anion generation and elastase release. Compared to the untreated group, * indicates p<0.05, ** indicates p<0.01, and *** indicates p<0.001.

FIG. 3 shows the cell viability of human neutrophils treated with the new compounds of the present invention.

FIG. 4 shows SSC and SSE inhibiting the production of reactive oxygen species (ROS) in neutrophils activated by a formyl peptide receptor 1 (FPR1) agonist.

FIG. 5 indicates that SSC blocks the binding of FPR1 agonist (such as Formyl-NIe-Leu-Phe-NIe-Tyr-Lys (fNLFNYK)) to FPR1 in a dose-dependent manner.

FIG. 6 indicates that SSE blocks the binding of FPR1 agonist (such as fNLFNYK) to FPR1 in a dose-dependent manner.

FIG. 7 shows the growth rates of human hair dermal papilla cells (HHDPC) cultured in a cultural medium without containing fetal bovine serum (0% FBS) after treatment of the novel compounds of the present invention. Compared with the control group (DMSO), * indicates p<0.05.

FIG. 8 shows the growth rates of human hair dermal papilla cells (HHDPC) cultured in a cultural medium containing 5% fetal bovine serum (FBS) after treatment of the novel compounds of the present invention. Compared with the control group (DMSO), * indicates p<0.05.

FIG. 9 illustrates the experimental procedures for testing the effects of the novel compounds from the present invention on improving acute respiratory distress syndrome (ARDS). Symbol description: % indicates administration; * indicates ARDS induction; ! indicates sacrifice.

FIG. 10 shows the hematoxylin and eosin (H&E) stained tissue sections of the lungs from experimental animals.

FIG. 11 shows the quantitative results of stained lung tissue sections from experimental animals. FIG. 11A is the analysis of the Ly6G protein; FIG. 11B is the analysis of elastase release (Elastase); FIG. 11C is the analysis of 4-hydroxy-2-nonenal (4-HNE⁺); FIG. 11D is the analysis of citrullinated histone H3 (CitH3⁺); FIG. 11E is the analysis of interleukin-1ß (IL-1B). * Indicates p<0.05 compared to the untreated group.

FIG. 12 shows the results of biochemical analysis of blood from experimental animals (glutamic oxaloacetic transaminase (GOT), glutamic pyruvic transaminase (GPT), creatinine (CRE), blood urea nitrogen (BUN)). Compared with the untreated group, * indicates p<0.05.

FIG. 9 illustrates the experimental procedures for testing the effects of the novel compounds from the present invention on improving liver injuries. Symbol description: % indicates administration; * indicates acute liver injury (ALI) induction; ! indicates sacrifice.

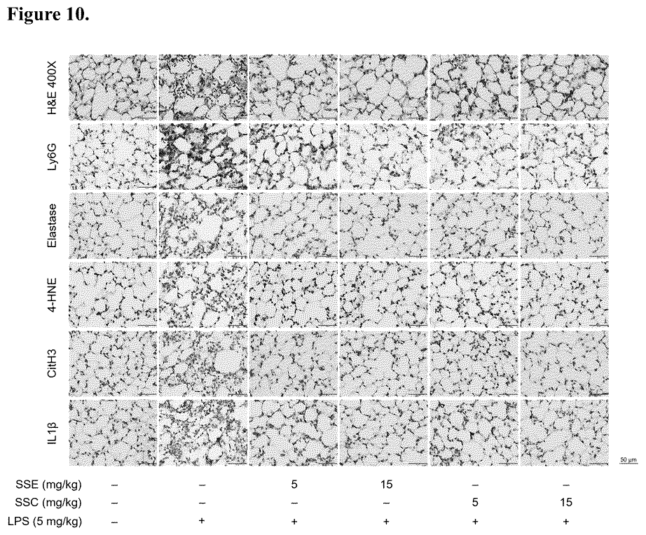

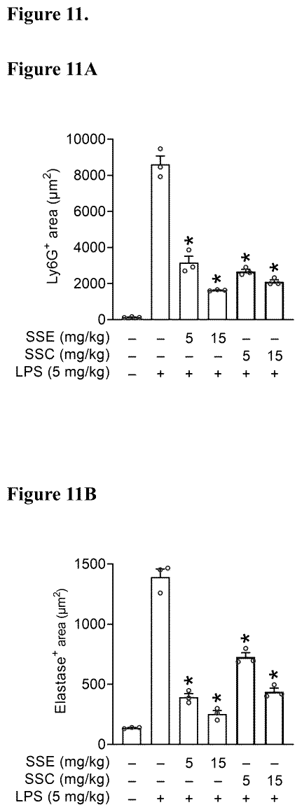

FIG. 10 shows the hematoxylin and eosin (H&E) stained tissue sections of the livers of experimental animals.

FIG. 15 shows the results of biochemical analysis of blood from experimental animals (glutamic oxaloacetic transaminase (GOT), glutamic pyruvic transaminase (GPT), creatinine (CRE), blood urea nitrogen (BUN)). Compared with the untreated group, * indicates p<0.05.

### DETAIL DESCRIPTION OF THE INVENTION

The present invention is a compound having a skeleton comprising a formula (I):

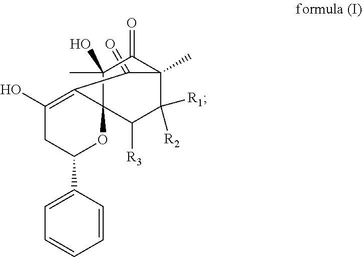

wherein, while R₃ is hydrogen (H), R₁ and R₂ together form a sesquiterpenoid; while R₂ is C1-C6 alkyl group, R₁ and R₃ together form a sesquiterpenoid.

Preferably, the compound of the present invention with a formula (I) includes

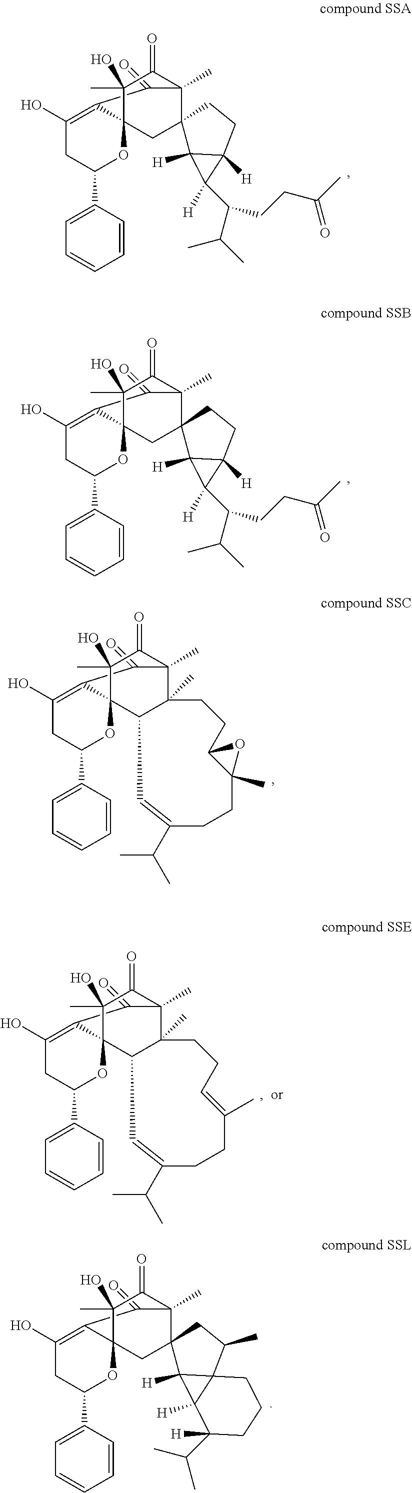

The present invention is related to a compound having a skeleton comprising a formula (II):

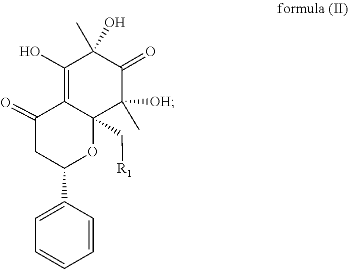

wherein, R₁ is a sesquiterpenoid.

Preferably, the compound of the present invention with a formula (II) is

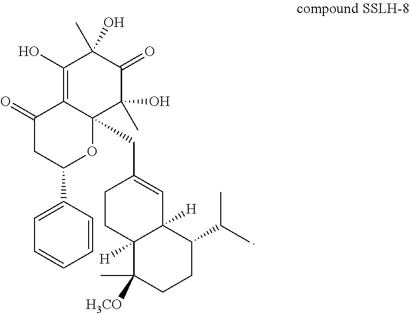 [SMILES: CO[C@]1(C)CC[C@@H](C(C)C)[C@@H]2C=C(C[C@]34O[C@H](c5ccccc5)CC(=O)C3=C(O)[C@](C)(O)C(=O)[C@]4(C)O)CC[C@@H]21]

The present invention is related to a compound having a skeleton comprising a formula (III):

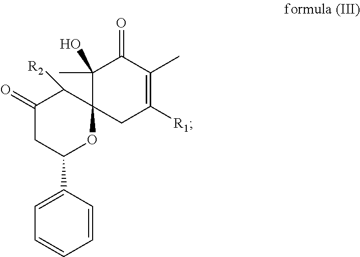

wherein, R₁ is a sesquiterpenoid, R₂ is hydrogen (H), acetoxy group, acetyl group, hydroxy group, methyl group, formyl group, formic acid group, methyl ester group, or halogen.

Preferably, the compound of the present invention with a formula (III) is

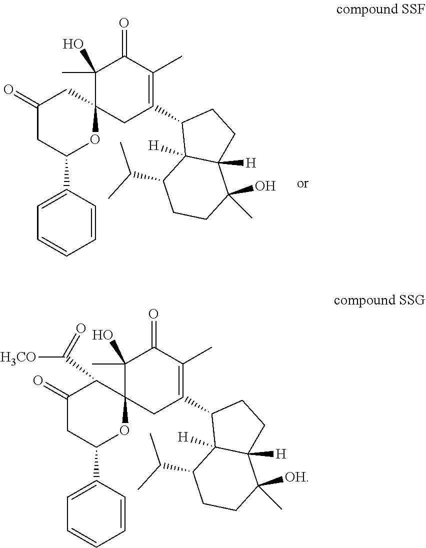 [SMILES: CC1=C([C@@H]2CC[C@H]3[C@H]2[C@@H](C(C)C)CC[C@]3(C)O)C[C@]2(CC(=O)C[C@@H](c3ccccc3)O2)[C@@](C)(O)C1=O ; COC(=O)[C@H]1C(=O)C[C@@H](c2ccccc2)O[C@@]12CC([C@@H]1CC[C@H]3[C@H]1[C@@H](C(C)C)CC[C@]3(C)O)=C(C)C(=O)[C@]2(C)O]

The present invention relates to a pharmaceutical composition for promoting hair growth, the pharmaceutical composition comprises a compound having formula (I), formula (II), or formula (III), or its medically acceptable salt, wherein, when the compound has a structure of formula (I), while R₃ is hydrogen (H), R₁ and R₂ together form a sesquiterpenoid; while R₂ is C1-C6 alkyl group, R₁, and R₃ together form a sesquiterpenoid; When the compound has the structure of formula (II), R₁ is sesquiterpenoid; When the compound has a structure of formula (III), R₁ is sesquiterpenoid, and R₂ is a hydrogen (H), acetoxy group, acetyl group, hydroxy group, methyl group, formyl group, formic acid group, methyl ester group, or halogen.

The present invention relates to a pharmaceutical composition for inhibiting inflammatory disorders, the pharmaceutical composition comprises a compound having formula (I), formula (II), or formula (III), or its medically acceptable salt, wherein, when the compound has a structure of formula (I), while R₃ is hydrogen (H), R₁ and R₂ together form a sesquiterpenoid; while R₂ is C1-C6 alkyl group and R₁ and R₃ together form a sesquiterpenoid; When the compound has the structure of formula (II), R₁ is sesquiterpenoid; When the compound has a structure of formula (III), R₁ is sesquiterpenoid, and R₂ is a hydrogen (H), acetoxy group, acetyl group, hydroxy group, methyl group, formyl group, formic acid group, methyl ester group, or halogen.

Preferably, the compound of the present invention with a formula (I) includes

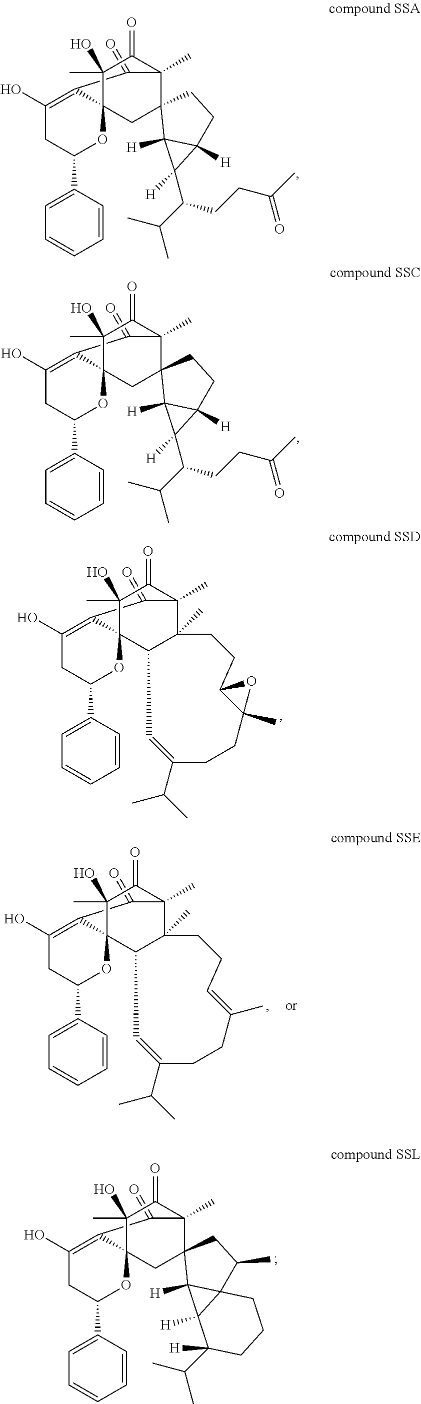

the compound with a formula (II) is

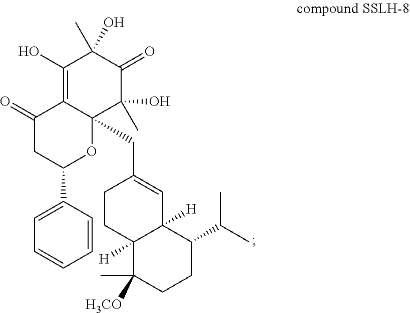 [SMILES: CO[C@]1(C)CC[C@@H](C(C)C)[C@@H]2C=C(C[C@]34O[C@H](c5ccccc5)CC(=O)C3=C(O)[C@](C)(O)C(=O)[C@]4(C)O)CC[C@@H]21]

and the compound with a formula (III) is

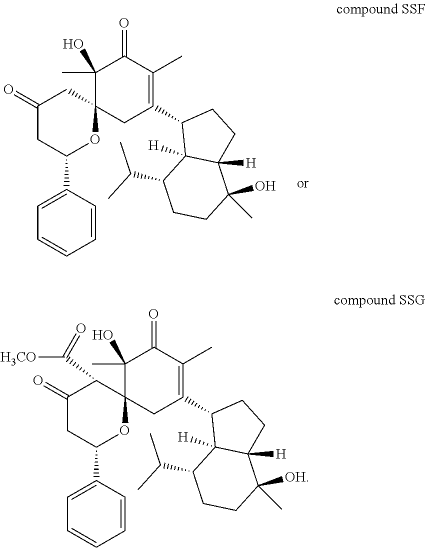 [SMILES: CC1=C([C@@H]2CC[C@H]3[C@H]2[C@@H](C(C)C)CC[C@]3(C)O)C[C@]2(CC(=O)C[C@@H](c3ccccc3)O2)[C@@](C)(O)C1=O ; COC(=O)[C@H]1C(=O)C[C@@H](c2ccccc2)O[C@@]12CC([C@@H]1CC[C@H]3[C@H]1[C@@H](C(C)C)CC[C@]3(C)O)=C(C)C(=O)[C@]2(C)O]

The present invention also relates to a method for promoting hair growth in a subject, comprising administering a compound having a formula (I), a formula (II) or a formula (III), or its pharmaceutically acceptable salts to the subject, wherein the subject is suffering from hair loss or having a need for hair growth.

The present invention also relates to a method for inhibiting inflammatory disorders in a subject, comprising administering a compound having a formula (I), a formula (II), or a formula (III), or its pharmaceutically acceptable salts to the subject, wherein the subject is suffering from inflammatory disorders.

In the present invention, the inflammatory disorders comprise lung injury or liver injury.

In the present invention, the subject of the present invention is human or mammal.

The pharmaceutical composition of the present invention can include a pharmaceutically acceptable excipient, particularly a predetermined solvent or oil, a pH regulator if necessary, and can also include a dispersing agent. Examples of solvents used in the present invention include, but are not limited to, water, ethanol, isopropanol, 1,3-butanediol, propylene glycol, glycerin, etc. Examples of oils used in the present invention are selected from the group consisting of, but are not limited to, corn oil, sesame oil, flaxseed oil, cottonseed oil, soybean oil, peanut oil, mono-glycerides, di-glycerides, tri-glycerides, mineral oil, squalene, jojoba oil, olive oil, evening primrose oil, borage oil, grape seed oil, coconut oil, sunflower oil, shea butter, and any combinations thereof.

In a preferred manner, the solvents and oils can be used alone or in any combination thereof.

Examples of useful dispersants that are beneficial to the present invention can include, but are not limited to, lecithin, organic monoglycerides, sorbitan fatty acid esters, polyoxyethylene fatty acid esters, sorbitan stearate, etc. These raw materials can also be used alone or in any combination thereof.

In the present invention, the pharmaceutical composition is preferably administered orally or is prepared as external preparations; examples of the external preparations can include but are not limited to, creams, ointments, gels, wash lotions or patches, etc., or inhalants, aerosols, suppositories, etc.

When the drug is used as an external preparation, an appropriate external skin preparation can be used as a base material, and an aqueous solution, a non-aqueous solvent, a suspension, an emulsion, or a lyophilized preparation, etc., and can be used and sterilized according to known methods. The compositions in the form of gels, creams, and ointments can be prepared according to the form of the composition by using known methods and by the addition of known softeners, emulsifiers, and thickeners, or other materials known in the art. The gel-form composition can be prepared, for example, by the addition of a softener such as trimethylolpropane, polyethylene glycol, and glycerol, for example, a solvent of propylene glycol, ethanol and isocetyl alcohol, and pure water.

In the present invention, liver injury includes but is not limited to, liver injury (including but not limited to acute liver injury or chronic liver injury), liver diseases such as hepatitis (including but not limited to alcoholic hepatitis, drug-induced hepatitis, steatohepatitis, viral hepatitis, chronic hepatitis), fatty liver, liver fibrosis, liver cirrhosis, liver cancer and other liver diseases caused by liver injury.

In the present invention, lung injury includes but is not limited to acute respiratory distress syndrome (ARDS).

### EMBODIMENTS

The present embodiment only describes the best embodiment and is not intended to limit the present invention.

### 1. Extraction of Compounds

In the present invention, leaf samples of Syzygium simile (10.8 kg) were collected and dried (5.5 kg). The dried leaf samples were soaked in methanol at room temperature for 3 days, repeated a total of 3 times. The resulting extract was reduced and compressed to obtain a methanol extract (650 g). The methanol extract was divided into n-hexane/water (1:1, v/v) to obtain the n-hexane layer (220 g) and the aqueous layer (22.5 L). 180 g of n-hexane layer was taken and separated by gravity column chromatography, and silica gel (SiliaFlash®) was used as the stationary phase, and the gradient extraction was carried out by the solvent system n-hexane/acetone (98:2, v/v), the proportion of acetone was gradually increased, and finally the high-polar part was extracted by methanol, and a total of 14 fractions were obtained. Subsequently, with methods of medium-pressure liquid chromatography (MPLC) and preparative thin-layer chromatography (preparative TLC) were used, compound simisyzygin A (SSA), compound simisyzygin B (SSB), compound simisyzygin E (SSE), simicadinene A (SSLH-8) were obtained from fraction 10, compound simisyzygin C (SSC), compound simisyzygin E (SSE), compound simisyzygin L (SSL) were obtained from fraction 7, and compound simisyzygin F (SSF), compound simisyzygin G (SSG) were obtained from fraction 11. The structures of those compounds were further confirmed by mass spectrometer, ultraviolet light analyzer, infrared light analyzer, polarimeter, circular dichroism spectrophotometer, X-ray diffractometer, etc.

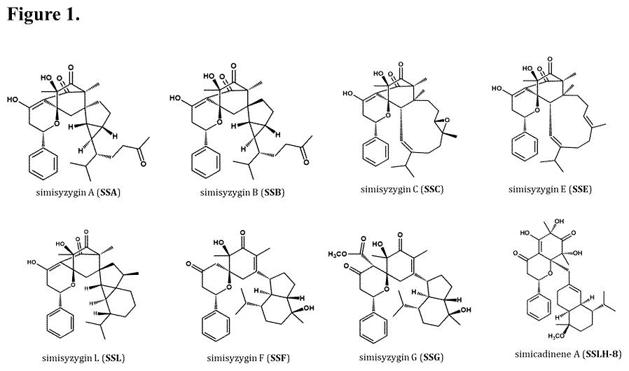

The structures of the compounds above are shown in FIG. 1.

### 2. Superoxide Anion and Elastase Release Inhibition Assay

The present invention evaluated the anti-inflammatory activity of the aforementioned compounds through superoxide anion and elastase release inhibition assays.

First, the preparation of neutrophils.

Blood was collected (approximately 50 mL) from healthy blood donors (aged 20-35 years; with regular lifestyle habits and no medication for at least one week) who voluntarily assisted or were interested in the project. The blood was drawn from the elbow vein using sterile vacuum tubes and then mixed with an equal volume of 3% dextran solution. The mixture was allowed to stand for sedimentation of red blood cells. The supernatant containing neutrophils was carefully layered onto a centrifuge tube containing Ficoll-Paque cell separation solution. After centrifugation, different blood cells were separated by density gradient, resulting in neutrophils and a small amount of red blood cells pelleted at the bottom. The remaining red blood cells were lysed using different concentrations of sodium chloride (NaCl) solution, exploiting the osmotic fragility differences to retain the desired neutrophils. Unless otherwise specified, neutrophils were suspended in Hank's balanced salt solution (HBSS) containing 1 mM calcium chloride (CaCl₂)) and 1 mM magnesium chloride (MgCl₂) for the anti-inflammatory experiments.

The evaluation of the anti-inflammatory effects of the compounds in the present invention on neutrophils was conducted using respiratory burst assays and degranulation assessments.

Respiratory burst mainly produces superoxide anions (O₂^{·-}) and reactive oxygen species (ROS), the detection of superoxide anion (O₂^{·-}) was performed by adding the compounds to a neutrophil suspension (6*10⁵ cells/mL) containing 0.6 mg/mL of ferricytochrome c at 37° C. for 2 minutes and then treated with 1 μg/mL cytochalasin B (CB) for 3 minutes. Then, N-formyl-L-methionyl-L-leucinoyl-L-phenylalanine (fMLF) was used as a stimulant to activate the cells for 10 minutes. The absorbance value was then measured at a wavelength of 550 nm using a UV spectrophotometer.

In the elastase release experiment, a neutrophil suspension (6*10⁵ cells/mL) containing 100 UM methoxysuccinyl-ala-ala-prolineate-valine-p-nitroanilide (MeOSuc-Ala-Ala-Pro-Val-p-nitroanilide) was incubated with compound at 37° C. for 2 minutes. Cells were then treated with 0.5 μg/mL cytochalasin B (CB) for 3 minutes, and the neutrophils were activated by adding 0.1 μM fMLF for 10 minutes. The absorbance value was measured at 405 nm using a UV spectrophotometer. Experimental results were expressed as percentage inhibition.

The results, as shown in Table 1, indicate that all compounds significantly inhibited neutrophil inflammation, with compounds SSC and SSE showing the most potent effects.

TABLE 1

| | Superoxide anion | | | Elastase release | | |
|---|---|---|---|---|---|---|
| Compounds | IC₅₀ (μM) | Inh % | | IC₅₀ (μM) | Inh % | |
| SSA | 0.39 ± 0.14 | 102.02 ± 0.90 | *** | 0.76 ± 0.22 | 120.13 ± 6.41 | *** |
| SSB | 0.83 ± 0.18 | 102.53 ± 0.48 | *** | 2.04 ± 0.59 | 109.09 ± 6.84 | *** |
| SSC | 0.04 ± 0.02 | 100.17 ± 0.46 | *** | 0.16 ± 0.03 | 109.63 ± 4.05 | *** |
| SSE | 0.15 ± 0.02 | 104.05 ± 0.48 | *** | 0.35 ± 0.10 | 116.41 ± 3.51 | *** |
| SSF | >10 | 33.73 ± 1.21 | *** | >10 | 47.64 ± 3.96 | *** |
| SSG | 4.38 ± 0.37 | 86.26 ± 3.70 | *** | 4.44 ± 0.26 | 74.28 ± 1.64 | *** |
| SSL | — | — | | 0.59 ± 0.10 | 117.33 ± 2.10 | *** |
| SSLH-8 | 0.59 ± 0.12 | 100.87 ± 1.34 | *** | 1.16 ± 0.24 | 112.79 ± 2.78 | *** |

Human neutrophils were treated with 0.1% dimethyl sulfoxide (DMSO, as the control group) or compounds (as the experimental group) for 5 minutes, followed by the addition of fMLF/CB for 10 minutes.
Percentage of inhibition (Inh %) at 3 μM.
Results are presented as mean ± S.E.M. (n = 6).
*** P < 0.005 compared with the control.
IC₅₀ means concentration necessary for 50% inhibition.

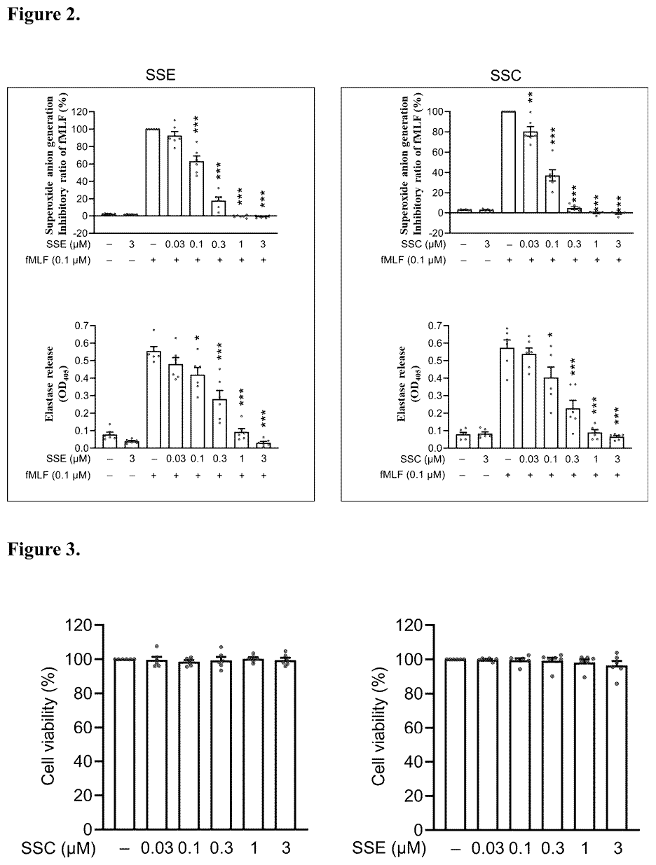

The results of different concentrations (0.03 μM, 0.1 μM, 0.3 μM, 1 μM, 3 μM) of the compounds inhibiting superoxide anion and elastase release are shown in FIG. 2. The results indicate that SSC and SSE at all concentrations inhibited superoxide anion and elastase release in a dose-dependent manner, with significant inhibition observed starting from 0.1 μM for each compound.

Lactate dehydrogenase (LDH) is normally present in the cytoplasm of cells. When cells die, LDH is released into the extracellular space. By measuring the extracellular LDH concentration, it can be used to determine whether a compound has cytotoxicity.

Neutrophil suspension (6*10⁵ Cell/mL) was incubated with compounds or TritonX-100 (as total LDH release) at 37° C. for 15 minutes. The samples were centrifuged at 200×g for 8 minutes at 4° C., followed by collection of the supernatant for LDH content measurement using an LDH ELISA kit.

The cell viability of neutrophils treated with various compounds from the present invention is shown in FIG. 3, indicating that none of the compounds exhibit cytotoxicity.

The effect of the compounds on reactive oxygen species (ROS) was analyzed using chemiluminescence. Neutrophil suspensions (7*10⁵ cells/mL) containing 37.5 μM luminol in a 96-well white plate were pre-warmed at 37° C. for 5 minutes. The test compounds were then added and incubated for 5 minutes. Neutrophils were stimulated to produce ROS by adding N-Formylmethionine-leucyl-phenylalanine (fMLF; 0.1 μM) for 6 minutes. The production of ROS was measured using a luminescence plate reader.

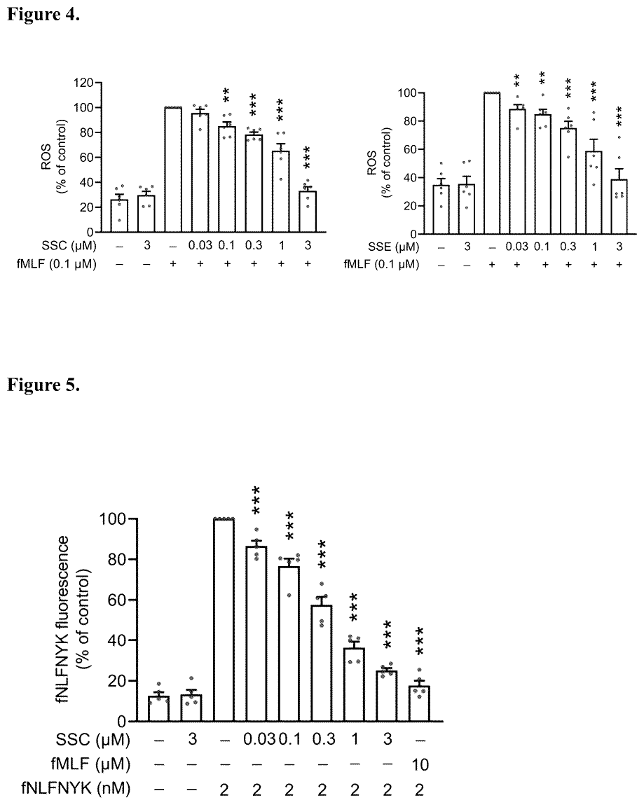

The results of different concentrations (0.03 μM, 0.1 μM, 0.3 μM, 1 μM, 3 μM) of the compounds on inhibiting ROS are shown in FIG. 4. The results indicate that SSC and SSE at all concentrations have the ability to inhibit ROS formation by neutrophils, with a dose-dependent manner.

Neutrophils (2*10⁶ Cell/mL) were suspended in Hank's balanced salt solution (HBSS) without calcium chloride (CaCl₂)) and magnesium chloride (MgCl₂) for the experiment. The test compounds were added at 4° C. for 10 minutes, followed by the addition of Formyl-NIe-Leu-Phe-NIe-Tyr-Lys (fNLFNYK; 2 nM) for 20 minutes. The fluorescence expression of fNLFNYK on the cells was measured using flow cytometry.

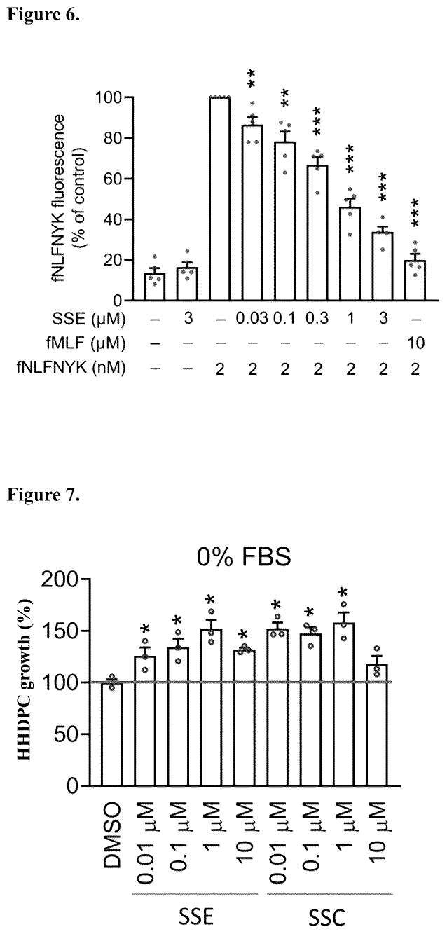

Based on the experimental results shown in FIGS. 5 and 6, SSC and SSE at various concentrations (0.03 μM, 0.1 μM, 0.3 μM, 1 μM, 3 μM) exhibit a dose-dependent reduction in the binding of fNLFNYK (2 nM) to FPR1. This demonstrates that SSC and SSE have the ability to bind to FPR1 on neutrophils.

The experimental results indicate that SSC and SSE can bind to FPR1 on neutrophils, thereby influencing the neutrophil inflammatory response, including releasing superoxide anions, elastase, and ROS produced by neutrophils. Based on the above data, SSC and SSE have the potential to be developed as therapeutic agents for immune-related inflammatory diseases.

### 3. The Evaluation of Hair Growth-Promoting Efficacy

Human hair dermal papilla cells (HHDPC) were seeded in 24-well plates at a density of 2*10³ cells per well and cultured at 37° C. in C-MSCM medium. After 24 hours of incubation, the old C-MSCM medium was aspirated.

Each well was replenished with 400 μL of C-MSCM medium without fetal bovine serum (FBS) or with 5% FBS, followed by adding either the control group solution or different concentrations of the test compounds. The plates were then incubated in a 37° C. incubator for 48 hours. After incubation, 20 μL of WST-1 was added into each well, and the plates were further incubated at 37° C. for 2 hours. Absorbance at 450 nm and 620 nm were measured.

The results, as shown in FIG. 7, indicate that under 0% FBS conditions, SSE at concentrations ranging from 0.01 to 10 μM promotes the proliferation of HHDPC, with the best effect observed at 1 μM. For SSC, the results show that concentrations between 0.01 and 1 μM effectively promote HHDPC proliferation.

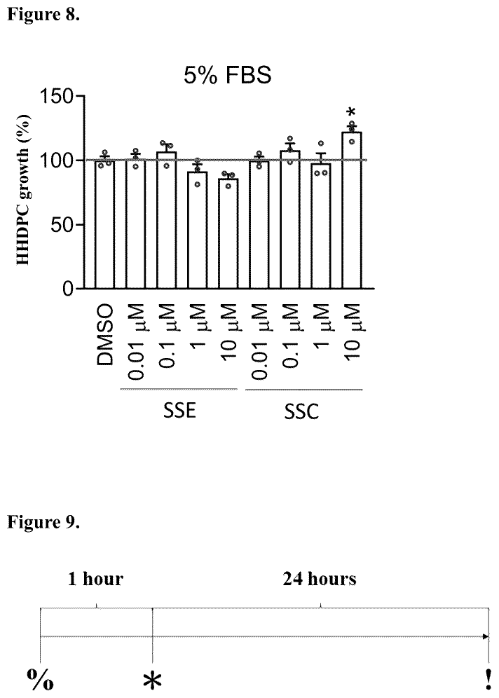

Under 5% FBS conditions (FIG. 8), no promotion of HHDPC proliferation was observed with SSE. However, the results for SSC indicate that 10 μM concentration of SSC had a proliferative effect on HHDPC cells.

SSE and SSC effectively promote the proliferation of HHDPC and can develop as hair growth raw materials.

### 4. Evaluation of the Effect on Improving Acute Respiratory Distress Syndrome (ARDS)

All animal experiments were approved by the Institutional Animal Care and Use Committee (IACUC) of Chang Gung University.

18 male BALB/c mice, aged 7-10 weeks and weighing 20-25 grams, were randomly divided into six groups as follows: negative control group (untreated, uninduced; n=3), lipopolysaccharides (LPS) induced control (untreated, ARDS induced by LPS; n=3), low-dosage SSC group (administered 5 mg/kg body weight SSC, ARDS induced by LPS; n=3), high-dosage SSC group (administered 15 mg/kg body weight SSC, ARDS induced by LPS; n=3), low-dosage SSE group (administered 5 mg/kg body weight SSE, ARDS induced by LPS; n=3), and high-dosage SSC group (administered 15 mg/kg body weight SSE, ARDS induced by LPS; n=3).

As shown in FIG. 9, at the beginning of the experiment, except for the negative control group and the LPS control group, different compounds of the present invention were first given intravenously (I.V.) to the experimental animals according to their groups, respectively. One hour after different compounds administration, all experimental animals except for the negative control group were administered 5 mg/kg body weight of LPS using a lung administration sprayer (purchased from Shanghai Yuyan Instruments Co., Ltd.). All experimental animals were euthanized 24 hours after ARDS induction, blood was collected and centrifuged, the supernatant was taken for subsequent biochemical value detection, and lungs were collected under 21 cm H₂O column pressure, half of which was used for Hematoxylin & Eosin (H&E) staining, and the other half was frozen at −80° C.

The lung tissue staining results, as shown in FIG. 10, demonstrate that the compounds of the present invention can effectively improve the occurrence or reduce the severity of ARDS in a dose-dependent manner.

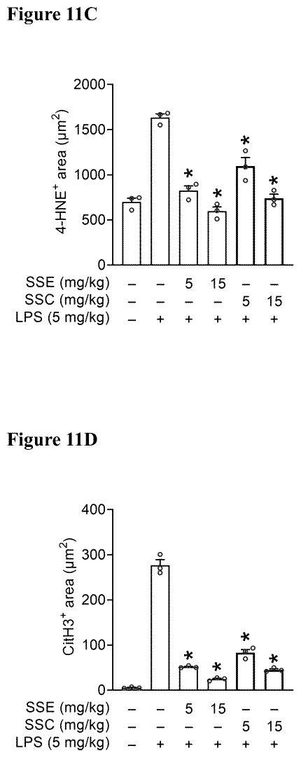

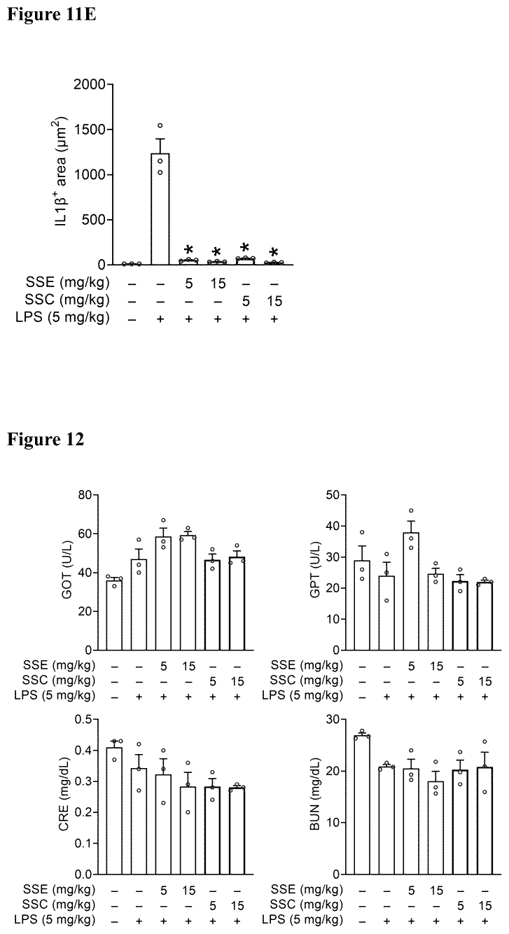

The quantitative results of lung tissue staining and blood biomarkers, including Ly6G protein, interleukin-1B (IL-1B), elastase release, 4-hydroxy-2-nonenal (4-HNE), and citrullinated histone H3 (CitH3⁺), as shown in FIGS. 11A to 11E, indicate that the compounds can effectively improve the occurrence or reduce the severity of ARDS in a dose-dependent manner.

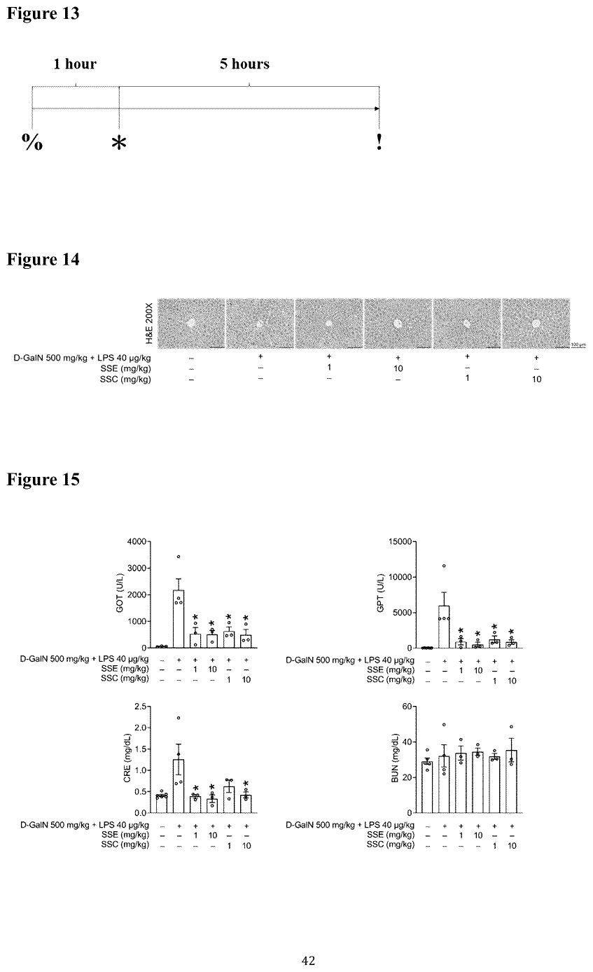

The blood analysis results, as shown in FIG. 12, indicate that in the experimental animals treated with the compounds of the present invention, the levels of glutamic oxaloacetic transaminase (GOT), glutamic pyruvic transaminase (GPT), creatinine (CRE), and blood urea nitrogen (BUN) did not show any abnormal elevation. All levels were within the normal range, indicating no harm to the liver or kidneys.

### 5. Evaluation of the Effect on Improving Acute Liver Injury (ALI)

All animal experiments were approved by the Institutional Animal Care and Use Committee (IACUC) of Chang Gung University.

As shown in FIG. 13, 18 male BALB/c mice, aged 7-10 weeks and weighing 20-25 grams, were randomly divided into six groups as follows: vehicle group (saline, without LPS+D-galactosamine (D-GalN) induction, without compounds administration), LPS+D-GalN control (LPS, 40 μg/kg body weight; D-GalN, 500 mg/kg body weight, without compounds administration), LPS+D-GalN+SSC low-dosage group (LPS, 40 μg/kg body weight; D-GalN, 500 mg/kg body weight; SSC, 1 mg/kg-body weight), LPS+D-GalN+SSC high-dosage group (LPS, 40 μg/kg body weight; D-GalN, 500 mg/kg body weight; SSC, 10 mg/kg-body weight), LPS+D-GalN+SSE low-dosage group (LPS, 40 μg/kg body weight; D-GalN, 500 mg/kg body weight; SSE, 1 mg/kg-body weight) and LPS+D-GalN+SSE high-dosage group (LPS, 40 μg/kg body weight; D-GalN, 500 mg/kg body weight; SSE, 10 mg/kg-body weight). Each mouse was intravenously injected with different concentrations of SSC or SSE to the corresponding group for 1 hour, followed by intraperitoneal injection of saline or LPS+D-GalN for another 5 hours. Blood samples were collected and analyzed for levels of glutamate pyruvate transaminase (GPT), glutamate oxaloacetic transaminase (GOT), creatinine (CRE), and urea nitrogen (BUN). Liver samples were collected, the right large section was used for H&E staining, and the remaining samples were frozen at −80° C.

The H&E staining results, as shown in FIG. 14, demonstrate that SSC and SSE can improve the disease condition of acute lung injury (ALI).

The blood biochemical analysis results, as shown in FIG. 15, indicate that D-GalN/LPS induced significant acute liver tissue injury, as evidenced by the elevated serum levels of GOT and GPT. Treatment with SSC and SSE effectively reduced the elevated levels of GOT and GPT.

While the invention has been described and exemplified in sufficient detail for those skilled in this art to make and use it, various alternatives, modifications, and improvements should be apparent without departing from the spirit and scope of the invention.

Those skilled in the art will readily understand and implement the objectives of the present invention, as well as achieve the aforementioned results and advantages. The sources of physiological signals, weight values, or gene sets used in this invention are provided for illustrative purposes and are not intended to limit the scope or application of the invention. Modifications or alternative uses generated by those skilled in the art when practicing or using this technology are encompassed within the spirit of the invention and defined by the scope of the claims.

What is claimed is:

1. A compound, which comprises a formula (I): 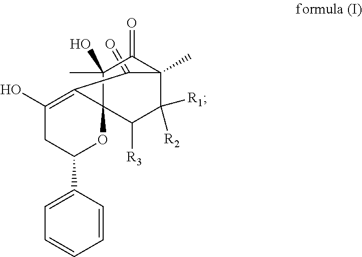
wherein, while R₃ is hydrogen (H), R₁ and R₂ together form a sesquiterpenoid; and
while R₂ is C1-C6 alkyl group, R₁ and R₃ together form a sesquiterpenoid.

2. The compound of claim 1, which comprises

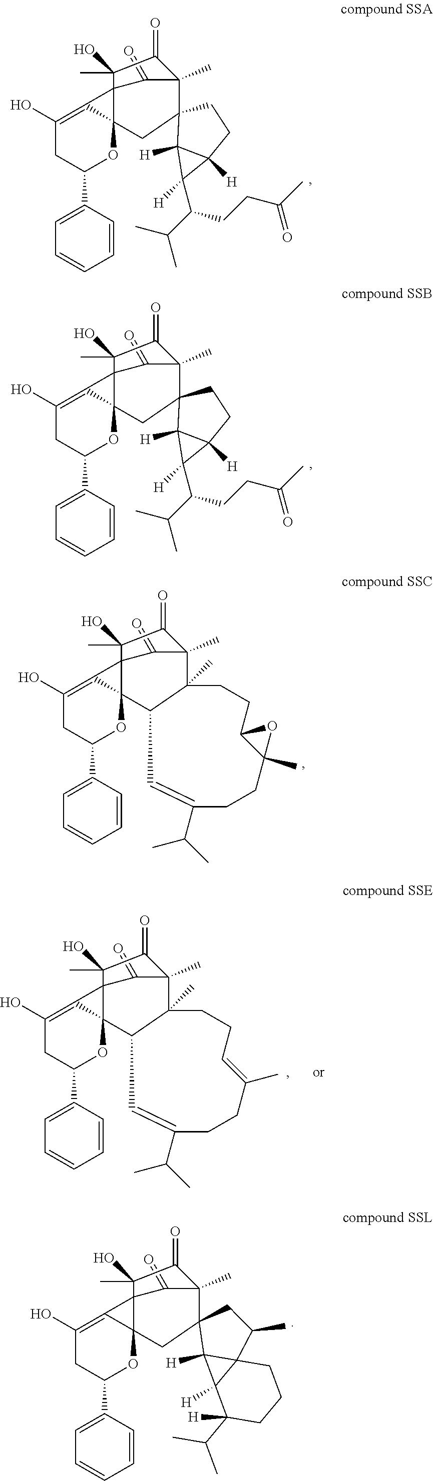

3. A compound, which comprises a formula (II): 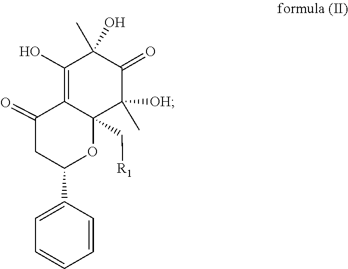
wherein, R₁ is a sesquiterpenoid.

4. The compound of claim 3, which comprises

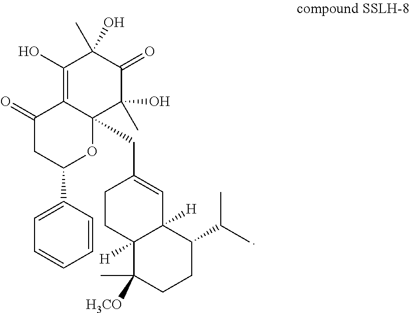 [SMILES: CO[C@]1(C)CC[C@@H](C(C)C)[C@@H]2C=C(C[C@]34O[C@H](c5ccccc5)CC(=O)C3=C(O)[C@](C)(O)C(=O)[C@]4(C)O)CC[C@@H]21]

5. A compound, which comprises a formula (III): 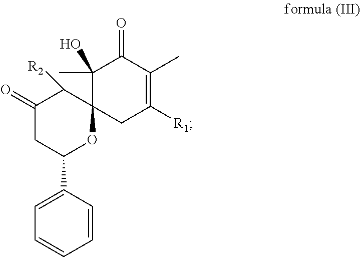
wherein, R₁ is a sesquiterpenoid, R₂ is hydrogen (H), acetoxy group, acetyl group, hydroxy group, methyl group, formyl group, formic acid group, methyl ester group, or halogen.

6. The compound of claim 5, which comprises

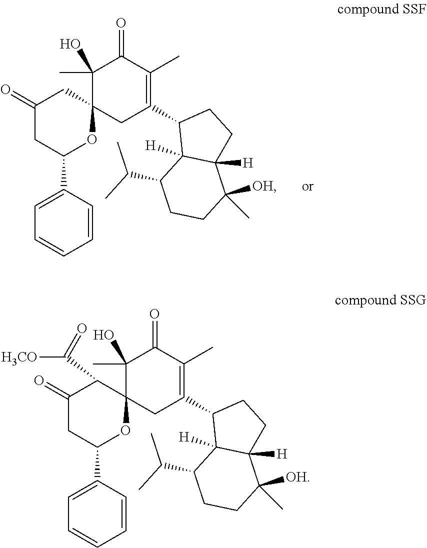 [SMILES: CC1=C([C@@H]2CC[C@H]3[C@H]2[C@@H](C(C)C)CC[C@]3(C)O)C[C@]2(CC(=O)C[C@@H](c3ccccc3)O2)[C@@](C)(O)C1=O ; COC(=O)[C@H]1C(=O)C[C@@H](c2ccccc2)O[C@@]12CC([C@@H]1CC[C@H]3[C@H]1[C@@H](C(C)C)CC[C@]3(C)O)=C(C)C(=O)[C@]2(C)O]

7. A pharmaceutical composition comprises a compound having a formula (I) of claim 1, or a compound having a formula (II) of claim 3, or a compound having a formula (III) of claim 5, or their medically acceptable salt thereof;
wherein, when the compound has a formula (I), while R₃ is hydrogen (H), R₁ and R₂ together form a sesquiterpenoid; while R₂ is C1-C6 alkyl group, R₁ and R₃ together form a sesquiterpenoid;
when the compound has a formula (II), R₁ is sesquiterpenoid; and
when the compound has a formula (III), R₁ is sesquiterpenoid, and R₂ is a hydrogen (H), acetoxy group, acetyl group, hydroxy group, methyl group, formyl group, formic acid group, methyl ester group, or halogen.

8. A method for promoting hair growth in a subject, comprising administering a pharmaceutical composition of claim 7 to the subject, wherein the subject is suffering from hair loss or having a need for hair growth.

9. A method for inhibiting inflammatory disorders in a subject, comprising administering a pharmaceutical composition of claim 7 to the subject, wherein the subject is suffering from inflammatory disorders.

10. The method of claim 9, wherein the inflammatory disorders comprise lung injury or liver injury.

11. The method of claim 9, wherein the inflammatory disorders comprise acute liver injury, chronic liver injury, alcoholic hepatitis, drug-induced hepatitis, steatohepatitis, viral hepatitis, chronic hepatitis, liver cancer, liver cirrhosis, liver fibrosis, fatty liver or acute respiratory distress syndrome (ARDS).
